(19) United States
(12) Patent Application Publication
(10) Pub. No.: US 2012/0046601 A1
(43) Pub. Date: Feb. 23, 2012

(54) Methods for delivering novel cell and cell-based compositions

(76) Inventor: GEORGE L. SING, NEW YORK, NY (US)

(21) Appl. No.: 13/199,020

(22) Filed: Aug. 17, 2011

Related U.S. Application Data
(60) Provisional application No. 61/402,075, filed on Aug. 23, 2010.

(51) Int. Cl. A61M 37/00

(52) U.S. Cl. 604/22

(57) ABSTRACT

The invention is directed to methods for delivering cells or cytokine-containing solutions into body tissue using ultrasound (sonoporation). Such methods utilize novel compositions including Amnion-derived Multipotent Progenitor cells (herein referred to as AMP cells) and Amnion-derived Cellular Cytokine Solution (herein referred to as ACCS), each alone or in combination with each other and/or other agents and/or other treatment modalities.

### CROSS-REFERENCE TO RELATED APPLICATIONS

This application claims priority under 35 USC§119(e) to U.S. Provisional Application No. 61/402,075, filed Aug. 23, 2010, the contents of which is incorporated herein by reference in its entirety.

### FIELD OF THE INVENTION

The field of the invention is directed to methods for delivering cells or cytokine-containing solutions into body tissue using ultrasound (sonoporation). Such methods utilize novel compositions including Amnion-derived Multipotent Progenitor cells (herein referred to as AMP cells) and Amnion-derived Cellular Cytokine Solution (herein referred to as ACCS), each alone or in combination with each other and/or other agents and/or other treatment modalities.

### DESCRIPTION OF RELATED ART

Bommannan, D., et al. (Pharm Res. 1992 April;9(4):559-64) describe the use of high-frequency ultrasound to enhance transdermal drug delivery.

Bommannan, D., et al. (Pharm Res. 1992 August;9(8):1043-7) report on the examination of the mechanism(s) of ultrasound-enhanced transdermal drug delivery.

Caskey, C. F., et al. (Conf Proc IEEE Eng Med Biol Soc. 2009;2009:134-6) describe ultrasound mediated drug delivery and the effect of microbubbles on a gel boundary.

Liang, H. D., et al. (Proc Inst Mech Eng H. 2010;224(2):343-61) review sonoporation, drug delivery, and gene therapy.

Liu, H. L., et al. (Radiology. 2010 May;255(2):415-25) report that blood-brain barrier disruption with focused ultrasound enhances delivery of chemotherapeutic drugs for glioblastoma treatment.

### BACKGROUND OF THE INVENTION

Most drug delivery occurs orally or by injection through the skin. Other common delivery methods involve transdermal delivery relying on absorption across the skin, or transmucosal delivery relying on absorption across the mucosa. While each delivery method is effective, each also has its drawbacks. For example, oral delivery of drugs has to take into consideration the acidic environment of the stomach and how that may impact the drug's effectiveness. In addition, this delivery route is systemic, so the drug is distributed to the entire body. Often, systemic delivery is not desirable. Injection (subcutaneous, intramuscular, intravenous, intraarterial, and the like) are all painful to some extent. Subcutaneous and intramuscular injections rely on diffusion to local capillaries for distribution to the body. All of these injections ultimately result in systemic distribution of the drug, which may not be desirable. Transdermal and transmuscosal delivery both rely on diffusion through tissue, which may be slow and inefficient, and again are systemic in nature. Most of the above methods are not perfect for targeted delivery to a particular site. It would be desirable, therefore, to have a delivery method which is easily targeted to a particular site, is not painful, is efficient and is capable of delivering suitable local doses by minimizing dilution. This would be particularly useful for therapies that are used to heal various types of wounds, including cutaneous wounds (burns, surgery, etc.) and deep wounds (soft tissue trauma, organ trauma, bone fracture), or even closed injuries such as closed traumatic brain injury where the brain has suffered, for example, a concussive (blow to head) or acceleration (shaken baby syndrome) injury but the skull itself is intact. Such injuries are difficult to treat for a variety of reasons, one being difficulty in delivering therapies across the boney skull directly to the injured brain tissue.

### BRIEF SUMMARY OF THE INVENTION

It is an object of the subject invention to provide a method for treating various types of injuries and wounds with wound healing agents utilizing ultrasound (sonoporation) to drive the therapeutic agent into the wounded/injured or diseased tissue.

A first aspect of the invention is a method of delivering cells into body tissue comprising a) mixing the cells with an aqueous medium; b) placing the aqueous medium/cell mixture on the body tissue; c) applying an ultrasound-delivering device to the body tissue; d) allowing the ultrasound-delivering device to emit ultrasound waves, wherein the ultrasound waves cause the cells to be delivered into the tissue. A particular embodiment is one in wherein the cells are AMP cells. Another particular embodiment is wherein the AMP cells are pooled AMP cells.

A second aspect of the invention is a method of delivering a cytokine-containing aqueous solution into body tissue comprising a) placing the cytokine-containing aqueous solution on the body tissue; c) applying an ultrasound-delivering device to the body tissue; d) allowing the ultrasound-delivering device to emit ultrasound waves, wherein the ultrasound waves cause the cytokine-containing aqueous solution to be delivered into the tissue. A particular embodiment is wherein the cytokine-containing solution is ACCS. Another particular embodiment is wherein the ACCS cells is pooled ACCS.

In particular embodiments of aspect one and two the tissue is epithelial tissue, connective tissue, muscle tissue, nervous tissue, or a combination thereof.

In a specific embodiment the epithelial tissue is on the external body surface or an internal body surface. In a particular and specific embodiment the external body surface is the epidermis and the internal body surface is any body cavity surface or internal organ surface.

Another specific embodiment is wherein the connective tissue is areolar connective tissue, reticular connective tissue, adipose connective tissue, dense regular connective tissue, dense irregular connective tissue, elastic connective tissue, hyaline cartilage, elastic cartilage, fibrocartilage, compact bone or cancellous bone, or combinations thereof.

Another specific embodiment is wherein the muscle tissue is skeletal muscle tissue, smooth muscle tissue or cardiac muscle tissue, or combinations thereof.

Another specific embodiment is wherein the nervous tissue is functional nervous tissue or supportive nervous tissue. In a specific embodiment the functional nervous tissue is comprised of neurons and the supportive nervous tissue is comprised of supporting cells selected from the group consisting of astrocytes, microglia, eppendymal cells, oligodendrocytes and Schwann cells. Another specific embodiment is a combination of functional and supportive nervous tissue.

In another embodiment of aspect one of the invention the aqueous medium is selected from the group consisting of water, normal saline, Ringer's solution, cell culture medium, aqueous gels, salves and suspensions.

In another embodiment of aspect one or two of the invention the ultrasound-delivering device is a wand, a probe, or other suitable ultrasound-delivering device.

In another embodiment of aspect one or two of the invention, the cells or cytokine-containing solution are delivered in combination with another agent and/or treatment modality.

A third aspect of the invention is wherein the AMP cells are treated such that they become genetically modified. In a specific embodiment the genetic modification is insertion of one or more genes into the cells. In a particular and specific embodiment the insertion of one or more genes results in the formation of an induced pluripotent cell or an immortalized cell.

Other features and advantages of the invention will be apparent from the accompanying description, examples and the claims. The contents of all references, pending patent applications and issued patents, cited throughout this application are hereby expressly incorporated by reference. In case of conflict, the present specification, including definitions, will control.

### Definitions

As defined herein “isolated” refers to material removed from its original environment and is thus altered “by the hand of man” from its natural state.

As defined herein, a “gene” is the segment of DNA involved in producing a polypeptide chain; it includes regions preceding and following the coding region, as well as intervening sequences (introns) between individual coding segments (exons).

As used herein, the term “protein marker” means any protein molecule characteristic of a cell or cell population. The protein marker may be located on the plasma membrane of a cell or in some cases may be a secreted protein.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning. In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning. Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “Amnion-derived Multipotent Progenitor cell” or “AMP cell” means a specific population of cells that are epithelial cells derived from the amnion. AMP cells have the following characteristics. They have not been cultured in the presence of any non-human animal materials, making them and cell products derived from them suitable for human clinical use as they are not xeno-contaminated. AMP cells are cultured in basal medium supplemented with human serum albumin. In a preferred embodiment, the AMP cells secrete the cytokines VEGF, Angiogenin, PDGF and TGFβ2 and the MMP inhibitors TIMP-1 and/or TIMP-2. The physiological range of the cytokine or cytokines in the unique combination is as follows: ˜5-16 ng/mL for VEGF, ˜3.5-4.5 ng/mL for Angiogenin, ˜100-165 pg/mL for PDGF, ˜2.5-2.7 ng/mL for TGFβ2, ˜0.68 μg mL for TIMP-1 and ˜1.04 μg/mL for TIMP-2. The AMP cells may optionally express Thymosin β4. AMP cells grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion-derived cells, from which AMP cells are isolated, will not react with antibodies to the stem/progenitor cell markers c-kit (CD117) and Thy-1 (CD90). Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved compositions and populations of cells.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such compositions and/or processes.

By the term “expanded”, in reference to cell compositions, means that the cell population constitutes a significantly higher yield of cells than is obtained using previous methods. For example, the level of cells per gram of amniotic tissue in expanded compositions of AMP cells is at least 50 and up to 150 fold higher than the number of cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of the cells.

As used herein, the term “passage” means a cell culture technique in which cells growing in culture that have attained confluence or are close to confluence in a tissue culture vessel are removed from the vessel, diluted with fresh culture media (i.e. diluted 1:5) and placed into a new tissue culture vessel to allow for their continued growth and viability. For example, cells isolated from the amnion are referred to as primary cells. Such cells are expanded in culture by being grown in the growth medium described herein. When such primary cells are subcultured, each round of subculturing is referred to as a passage. As used herein, “primary culture” means the freshly isolated cell population.

As used herein, the term “differentiation” means the process by which cells become progressively more specialized.

As used herein, the term “differentiation efficiency” means the percentage of cells in a population that are differentiating or are able to differentiate.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. Examples of methods of preparing conditioned media are described in U.S. Pat. No. 6,372,494 which is incorporated by reference in its entirety herein.

As used herein, the term “Amnion-derived Cellular Cytokine Solution” or “ACCS” means conditioned medium that has been derived from AMP cells or expanded AMP cells that have been cultured in basal media supplemented with human serum albumin. Amnion-derived cellular cytokine solution or ACCS has previously been referred to as “amnion-derived cytokine suspension”.

The term “physiological level” as used herein means the level that a substance in a living system is found, for example, in the circulatory system or in a particular microenvironment or biological niche in the living system, and that is relevant to the proper functioning of biochemical and/or biological processes.

As used herein, the term “pooled” means a plurality of compositions that have been combined to create a new composition having more constant or consistent characteristics as compared to the non-pooled compositions. For example, pooled ACCS has more constant or consistent characteristics compared to non-pooled ACCS. Examples of pooled compositions include “SP pools” (more than one ACCS collection/one placenta), “MP1 pools” (one ACCS collection/placenta, multiple placentas), and “MP2 pools” (more than one ACCS collection/placenta, multiple placentas).

The term “therapeutically effective amount” means that amount of a therapeutic agent necessary to achieve a desired physiological effect (i.e. treat wounds and injuries, cause tissue regeneration).

The term “lysate” as used herein refers to the composition obtained when cells, for example, AMP cells, are lysed and, optionally, the cellular debris (e.g., cellular membranes) is removed. Lysis may be achieved by mechanical means, by freezing and thawing, by sonication, by use of detergents, such as EDTA, or by enzymatic digestion using, for example, hyaluronidase, dispase, proteases, and nucleases. In some instances, it may be desirable to lyse the cells are retain the cellular membrane portion of the lysed cells.

As used herein, the term “pharmaceutically acceptable” means that the components, in addition to the therapeutic agent, comprising the formulation, are suitable for administration to the patient being treated in accordance with the present invention.

As used herein, the term “tissue” refers to an aggregation of similarly specialized cells united in the performance of a particular function.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

The term “ultrasound delivery” as used herein refers to the use of ultrasound as a delivery method to drive therapeutic agent(s) into tissue(s). For the purposes of this invention, the term “sonoporation” will mean the same thing as ultrasound delivery.

As used herein, the terms “a” or “an” means one or more; at least one.

As used herein, the term “adjunctive” means jointly, together with, in addition to, in conjunction with, and the like.

As used herein, the term “co-administer” can include simultaneous or sequential administration of two or more agents.

As used herein, the term “agent” means an active agent or an inactive agent. By the term “active agent” is meant an agent that is capable of having a physiological effect when administered to a subject. Non-limiting examples of active agents include growth factors, cytokines, antibiotics, cells, conditioned media from cells, etc. By the term “inactive agent” is meant an agent that does not have a physiological effect when administered. Such agents may alternatively be called “pharmaceutically acceptable excipients”. Non-limiting examples include time release capsules and the like.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

### Therapeutic Uses

Regenerative medicine—Wound and Injury—The methods and compositions of the invention are useful for treating all types of wounds and injuries including, but not limited to, cutaneous wounds, deep soft tissue wounds, bone fractures, brain injury, spinal cord injury, internal organ injury, joint injury, tendon and ligament injury, muscle injury, surgical wounds, etc. The methods and compositions of the invention are particularly useful for treating wounds and injuries that require more than one delivery of a therapeutic agent(s) but which are “closed” (i.e. a deep wound which has been sutured over, an injured joint, traumatic brain or spinal cord injury in which the bone is intact, and the like).

Regenerative medicine—Disease—The methods and compositions of the invention are useful for treating all types of degenerative disease including, but not limited to, bone degeneration, joint degeneration, nerve degeneration, muscle degeneration, liver degeneration, etc. The methods and compositions of the invention are particularly useful for treating such degenerative disease as they enable multiple deliveries of therapeutic agent(s) without opening the skin to enable direct delivery to the degenerating cells or tissue.

### Obtaining and Culturing of Cells

AMP cell compositions are prepared using the steps of a) recovery of the amnion from the placenta, b) dissociation of the epithelial cells from the amniotic membrane using a protease, c) culturing of the cells in a basal medium with the addition of a naturally derived or recombinantly produced human protein (i.e. human serum albumin) and no non-human animal protein; d) selecting AMP cells from the epithelial cell culture, and optionally e) further proliferation of the cells, optionally using additional additives and/or growth factors (i.e. recombinant human EGF). Details are contained in US Publication No. 2006-0222634-A1,which is incorporated herein by reference.

Culturing of the AMP cells—The cells are cultured in a basal medium. Such medium includes, but is not limited to, EPILIFE® culture medium for epithelial cells (Cascade Biologicals), OPTI-PRO™ serum-free culture medium, VP-SFM serum-free medium, IMDM highly enriched basal medium, KNOCKOUT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, UltraMDCK™ serum-free medium (all made by Cambrex), STEMLINE® T-cell expansion medium and STEMLINE® II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media. Preferably, the medium is IMDM highly enriched basal medium. Such media should either contain human protein or be supplemented with human protein. As used herein a “human protein” is one that is produced naturally or one that is produced using recombinant technology. In specific embodiments, the basal media is IMDM highly enriched basal medium and the human protein is human serum albumin at a concentration of at least 0.5% and up to 10%. In particular embodiments, the human serum albumin concentration is from about 0.5 to about 2%. The human serum albumin may come from a liquid or a dried (powder) form and includes, but is not limited to, recombinant human serum albumin, PLASBUMIN® normal human serum albumin and PLASMANATE® human blood fraction (both made by Talecris Biotherapeutics). In a most preferred embodiment, the cells are cultured using a system that is free of non-human animal products to avoid xeno-contamination.

Optionally, other factors are used. In one embodiment, human recombinant epidermal growth factor (hrEGF) at a concentration of between 0-1 μg/mL is used. In a preferred embodiment, the hrEGF concentration is around 10-20 ng/mL. All supplements are clinical grade.

In a specific embodiment, the following method is used to obtain selected AMP cells. The cells are plated into plastic tissue culture vessels (i.e. T75 flasks) immediately upon isolation from the amnion. After ˜1-5 days, preferably ˜1-3 days, and most preferably ˜2 days in culture, non-adherent cells are removed from the plastic tissue culture vessel and discarded and the adherent cells are kept. This attachment of cells to a plastic tissue culture vessel is the selection method used to obtain the desired population of AMP cells. Adherent and non-adherent AMP cells appear to have similar cell surface marker expression profiles but the adherent cells have the advantage of possessing greater viability than the non-adherent population of cells and are thus the desired population of AMP cells. Adherent AMP cells are cultured until they reach ˜13,000-700,000 cells/cm², preferably ˜53,000-500,000 cells/cm² and most preferably ˜120,000-300,000 cells/cm². At this point, the cultures are confluent or close to confluent. Suitable cells cultures will reach this number of cells between ˜5-14 days, preferably between 5-9 days. Attaining this criterion is an indicator of the proliferative potential of the AMP cells and cells that do not achieve this criterion are not selected for further analysis and use. Once the AMP cells reach ˜13,000-700,000 cells/cm², preferably ˜53,000-500,000 cells/cm² and most preferably ˜120,000-300,000 cells/cm², they are removed from the plastic tissue culture vessel and cryopreserved. This collection time point is called p0.

The AMP cells of the invention are characterized by assaying for secretion of physiologically relevant cytokines and growth factors. Suitable cells are those in which each cytokine or growth factor occurs in the physiological range of ˜5.0-16 ng/mL for VEGF, ˜3.5-4.5 ng/mL for Angiogenin, ˜100-165 pg/mL for PDGF, ˜2.5-2.7 ng/mL for TGFβ2, ˜0.68 μg/mL for TIMP-1 and ˜1.04 μg/mL for TIMP-2. The cells may optionally be assayed for Thymosin β4.

### Generation of Conditioned Medium

Generation of ACCS—The AMP cells of the invention can be used to generate ACCS. In one embodiment, the AMP cells are isolated as described herein and 1×10⁶ cells/mL are seeded into T75 flasks containing between 5-30 mL culture medium, preferably between 10-25 mL culture medium, and most preferably about 10 mL culture medium. The culture medium is preferably a basal medium (for example IMDM highly enriched basal medium) which is supplemented with human serum albumin as described above. The cells are cultured until confluent, the medium is changed and in one embodiment the ACCS is collected 1 day post-confluence. In another embodiment the medium is changed and ACCS is collected 2 days post-confluence. In another embodiment the medium is changed and ACCS is collected 4 days post-confluence. In another embodiment the medium is changed and ACCS is collected 5 days post-confluence. In a preferred embodiment the medium is changed and ACCS is collected 3 days post-confluence. In another preferred embodiment the medium is changed and ACCS is collected 3, 4, 5, 6 or more days post-confluence. Collected media is combined to create pools. A preferred pool is a SP pool. Skilled artisans will recognize that other embodiments for collecting ACCS from AMP cell cultures, such as using other tissue culture vessels, including but not limited to cell factories, flasks, hollow fibers, or suspension culture apparatus, or collecting ACCS from sub-confluent and/or actively proliferating cultures, are also contemplated by the methods of the invention. It is also contemplated by the instant invention that the ACCS be cryopreserved following collection. It is also contemplated by the invention that ACCS be lyophilized following collection. It is also contemplated by the invention that ACCS be formulated for sustained-release following collection. It is also contemplated that ACCS production be scaled up for generation of sufficient product for clinical testing and for commercialization. It is also contemplated by the invention that ACCS be irradiated. Skilled artisans are familiar with cryopreservation lyophilization, irradiation and sustained-release formulation methodologies.

Induced pluripotent cells—The AMP cells described herein may be treated such as to produce induced pluripotent cells (iPCs). Details on this can be found in PCT/US10/00122, which is incorporated herein by reference. Such iPCs are suitable for use in the methods of the invention described herein to treat, for example, wounds and injuries, or cause tissue regeneration.

Immortalized cells—The AMP cells described herein may be treated such as to produce immortalized AMP cells (I-AMP cells). Details on this can be found in U.S. Provisional Application No. 61/339,457, which is incorporated herein by reference. Such I-AMP cells are suitable for use in the methods of the invention described herein to treat, for example, wounds and injuries, or cause tissue regeneration.

The compositions of the invention can be prepared in a variety of ways depending on the intended use of the compositions. For example, a composition useful in practicing the invention may be a liquid comprising an agent of the invention, i.e. cells, for example, AMP cells, in solution, in suspension, or both (solution/suspension). The term “solution/suspension” refers to a liquid composition where a first portion of the active agent is present in solution and a second portion of the active agent is present in particulate form, in suspension in a liquid matrix. A liquid composition also includes a gel. The liquid composition may be aqueous or in the form of an ointment, salve, cream, or the like.

An aqueous suspension or solution/suspension useful for practicing the methods of the invention may contain one or more polymers as suspending agents. Useful polymers include water-soluble polymers such as cellulosic polymers and water-insoluble polymers such as cross-linked carboxyl-containing polymers. An aqueous suspension or solution/suspension of the present invention is preferably viscous or muco-adhesive, or even more preferably, both viscous and muco-adhesive.

Pharmaceutical Compositions—The present invention provides pharmaceutical compositions of cells, for example AMP cells or cytokine-containing solutions, for example, ACCS, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Treatment Kits—The invention also provides for an article of manufacture comprising packaging material and a pharmaceutical composition of the invention contained within the packaging material, wherein the pharmaceutical composition comprises compositions of cells, for example AMP cells, or cytokine-containing solutions, for example, ACCS. The packaging material comprises a label or package insert which indicates that the compositions of cells, for example AMP cells or cytokine-containing solutions, for example, ACCS, can be used to treat, for example, wounds and injuries, or cause tissue regeneration.

### Formulation, Dosage and Administration

Compositions of cells, for example AMP cells, or cytokine-containing solutions, for example, ACCS, may be administered to a subject to provide various cellular or tissue functions, for example, to treat, for example, wounds and injuries, or cause tissue regeneration. As used herein “subject” may mean either a human or non-human animal.

Such compositions may be formulated in any conventional manner using one or more physiologically acceptable carriers optionally comprising excipients and auxiliaries. Proper formulation is dependent upon the route of administration chosen. The compositions may be packaged with written instructions for their use in for example, treating wounds and injuries, or causing tissue regeneration. The compositions may also be administered to the recipient in one or more physiologically acceptable carriers. Carriers for the cells may include but are not limited to solutions of phosphate buffered saline (PBS) or lactated Ringer's solution containing a mixture of salts in physiologic concentrations.

Pharmaceutical compositions useful in the practice of certain embodiments of the invention include a therapeutically effective amount of an active agent with a pharmaceutically acceptable carrier. Such pharmaceutical compositions may be liquid, gel, ointment, salve, slow release formulations or other formulations.

In various embodiments, compositions of the invention can comprise a liquid comprising an active agent in solution, in suspension, or both. The term “suspension” herein includes a liquid composition wherein a first portion of the active agent is present in solution and a second portion of the active agent is present in particulate form, in suspension in a liquid matrix. As used herein, liquid compositions include gels.

One of skill in the art may readily determine the appropriate concentration, or dose, of the cells, for example, AMP cells, or cytokine-containing solution, for example, ACCS, for a particular purpose. The skilled artisan will recognize that a preferred dose is one which produces a therapeutic effect, such as treating wounds and injuries, or causing tissue regeneration, in a patient in need thereof. For example, AMP cells are prepared at a concentration of between about 1×10⁷-1×10⁸ cells/mL, preferably at about 2.5×10⁷-7.5×10⁷ cells/mL, and most preferably at about 5×10⁷ cells/mL. The volume of cell mixture administered will depend upon several variables and can only be determined by the attending physician at time of use. In addition, one of skill in the art may readily determine the appropriate concentration of ACCS for a particular purpose. A preferred dose is in the range of about 0.5-2000 μl/cm². Another preferred dose is in the range of about 5-1000 μl/cm². Another preferred dose is in the range of about 50-100 μl/cm². In a particularly preferred embodiment, it has been found that relatively small amounts of ACCS can accelerate wound healing, etc. Of course, proper doses of AMP cells ACCS will require empirical determination at time of use based on several variables including but not limited to the severity and type of disease, injury, disorder or condition being treated; patient age, weight, sex, health; other medications and treatments being administered to the patient; and the like. One of skill in the art will also recognize that number of doses (dosing regimen) to be administered needs also to be empirically determined based on, for example, severity and type of disease, injury, disorder or condition being treated. In a preferred embodiment, one dose is sufficient. Other preferred embodiments contemplate, 2, 3, 4, or more doses.

The present invention provides a method of treating wounds and injuries, or causing tissue regeneration, by administering to a subject cells, for example, AMP cells, or cytokine-containing solution, for example, ACCS, in a therapeutically effective amount. By “therapeutically effective amount” is meant the dose of cells, for example AMP cells, or cytokine-containing solution, for example, ACCS, which is sufficient to elicit a therapeutic effect. Thus, the concentration of cells, for example AMP cells, or cytokine-containing solution, for example, ACCS, in an administered dose unit in accordance with the present invention is effective in, for example, the treatment of wounds and injuries, or causing tissue regeneration.

In further embodiments of the present invention, at least one additional agent or treatment modality may be combined with the cells, for example AMP cells, or cytokine-containing solution, for example, ACCS, to enhance treatment of wounds and injuries, or causing tissue regeneration. Such agents or treatment modalities may include, for example, dietary supplementation or replacement, vitamins, intermediary metabolites, compounds or drugs that facilitate or retard specific metabolic pathways, enzyme replacement, cytokines, chemokines, antibodies, inhibitors, antibiotics, anti-fungals, anti-virals, immunosuppressive agents, and other cell types. In still another specific embodiment the other treatment modality is gene transfer. Inactive agents include carriers, diluents, stabilizers, gelling agents, delivery vehicles, ECMs (natural and synthetic), scaffolds, and the like. When the cells, for example AMP cells, or cytokine-containing solution, for example, ACCS, are administered conjointly with other pharmaceutically active agents, even less of the cells, for example AMP cells, or cytokine-containing solution, for example, ACCS, may be needed to be therapeutically effective.

Cells, for example AMP cells, or cytokine-containing solution, for example, ACCS, can be administered via a delivery device such as a tube, e.g., catheter, or a scope. An ultrasound probe may be inserted into the tube, catheter or scope. A non-limiting example would be inserting a scope through a joint capsule, placing the therapeutic agent (i.e. AMP cells and/or ACCS) into the synovial fluid within the capsule and using ultrasound to drive the therapeutic agent into the surrounding tissues. Specific, non-limiting examples of administering cells to subjects may also include administration by intravenous injection, intraarterial injection, intramuscular injection, intrathecal injection, epidural injection, or infusion each of which is accompanied by ultrasound administration.

The timing of administration of cells, for example AMP cells, or cytokine-containing solution, for example, ACCS, will depend upon the type and severity of the wounds and injuries being treated, or the tissue regeneration needed. In a preferred embodiment, the cells, for example AMP cells, or cytokine-containing solution, for example, ACCS, are administered as soon as possible after the injury, disease or disorder is diagnosed. In other preferred embodiments, the cells, for example AMP cells, or cytokine-containing solution, for example, ACCS, are administered more than one time following injury or diagnosis.

Also contemplated by the methods of the invention are compositions comprising differentiated cells. These differentiated cells are made by using AMP cells and treating them such that they become partially or fully differentiated cells, or combinations thereof. Such partially or fully differentiated cell compositions, or combinations thereof, are obtained by treating AMP cells with appropriate reagents and under appropriate conditions wherein the cells undergo partial or complete differentiation. Skilled artisans are familiar with conditions capable of effecting such partial or complete differentiation. The cells may be treated under differentiating conditions prior to use (i.e. prior to ultrasound delivery, etc.), simultaneously with use or post-use. In certain embodiments, the cells are treated under differentiation conditions before and during use, during and after use, before and after use, or before, during and after use.

Skilled artisans will recognize that any and all of the standard methods and modalities for treating injuries, diseases or disorders currently in clinical practice and clinical development are suitable for practicing the methods of the invention. Routes of administration, formulation, co-administration with other agents (if appropriate) and the like are discussed in detail elsewhere herein.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Preparation of AMP Cell Compositions

Recovery of AMP cells—AMP cells were dissociated from starting amniotic membrane using the dissociation agents PXXIII. The average weight range of an amnion was 18-27 g. The number of cells recovered per g of amnion was about 10-15×10⁶.

Method of obtaining selected AMP cells—Cells were plated immediately upon isolation from the amnion. After ˜2 days in culture non-adherent cells were removed and the adherent cells were kept. This attachment to a plastic tissue culture vessel is the selection method used to obtain the desired population of AMP cells. Adherent and non-adherent AMP cells appear to have a similar cell surface marker expression profile but the adherent cells have greater viability and are the desired population of cells. Adherent AMP cells were cultured in IMDM basal medium supplemented with 0.5% human serum albumin until they reached ˜120,000-150,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are not selected for further analysis and use. Once the AMP cells reached ˜120,000-150,000 cells/cm², they were collected and cryopreserved. This collection time point is called p0.

### Example 2

### Generation of ACCS

The AMP cells of the invention can be used to generate ACCS, including pooled ACCS. The AMP cells were isolated as described above and ˜1×10⁶ cells/mL were seeded into T75 flasks containing ˜10 mL culture medium as described above. The cells were cultured until confluent, the medium was changed and ACCS was collected 3 days post-confluence. Optionally, the ACCS is collected again after 3 days, and optionally again after 3 days. Collected media are combined to make pools. Skilled artisans will recognize that other embodiments for collecting ACCS from confluent cultures, such as using other tissue culture vessels, including but not limited to cell factories, flasks, hollow fibers, or suspension culture apparatus, etc. are also contemplated by the methods of the invention (see Detailed Description above). It is also contemplated by the instant invention that the ACCS be cryopreserved, lyophilized, irradiated or formulated for sustained-release following collection. It is also contemplated that ACCS be collected at different time points (see Detailed Description for details).

### Example 3

### Generation of Pooled ACCS

ACCS was obtained essentially as described above. In certain embodiments, ACCS was collected multiple times from an AMP cell culture derived from one placenta and these multiple ACCS collections were pooled together. Such pools are referred to as “SP pools” (more than one ACCS collection/one placenta). In another embodiment, AMP cell cultures were derived from several placentas, i.e. from 5 or 10 placentas. The AMP cells from each placenta were cultured and one ACCS collection from each culture was collected and then they were all pooled. These pools are termed “MP1 pools” (one ACCS collection/placenta, multiple placentas). In yet another embodiment, AMP cell cultures were derived from several placentas, i.e. from 5 or 10 placentas. The AMP cells from each placenta were cultured and more than one ACCS collection was performed from each AMP cell culture and then pooled. These pools are termed “MP2 pools” (more than one ACCS collection/placenta, multiple placentas).

### Example 4

### Evaluation of Ultrasound Delivery of AMP Cells, Immortalized AMP Cells, or iPCs made from AMP Cells in Animal Models of Wound Healing and Injury, and Tissue Regeneration

AMP cells, immortalized AMP cells or iPCs made from AMP cells are tested in various animal models of wound healing (see, for example, chronic wound model: Hayward P G, Robson M C: Animal models of wound contraction. In Barbul A, et al: Clinical and Experimental Approaches to Dermal and Epidermal Repair: Normal and Chronic Wounds. John Wiley & Sons, New York, 1990; spinal cord injury model: Constantini, S., and Young, W. (1994), J. Neurosurg. 80, 97-111; Anderson, A. J., et al., Journal of Neurotrauma 21 (12), 1831-1846; brain injury model: Finnie, J., Aust Vet J. 2001 September;79(9):628-33. Animal models of traumatic brain injury: a review).

AMP cells, immortalized AMP cells or iPCs made from AMP cells are tested in various animal models of tissue regeneration (see, for example, tendon and ligament: Carpenter, J. E. and Hankenson, K. D., 2004, Biomaterials 25(9):1715-1722, Animal Models for Tissue Engineering Applications; cartilage: Chu C R, et al., Tissue Eng Part B Rev. 2010 February;16(1):105-15, Animal models for cartilage regeneration and repair; bone: Pearce, S. G., European Cells and Materials Vol. 14. Suppl. 1, 2007 (page 42), Animal Models for Bone Repair; Matos, M. A., et al., Journal of Orthopaedic Surgery and Research 2008, 3:4, Histomorphometric evaluation of bone healing in rabbit fibular osteotomy model without fixation; muscle: De Bari, C., et al., J Cell Biol. 2003 Mar. 17; 160(6): 909-918. Skeletal muscle repair by adult human mesenchymal stem cells from synovial membrane).

### Example 5

### Evaluation of Ultrasound Delivery of ACCS in Animal Models of Wound Healing and Injury, and Tissue Regeneration

ACCS is tested in various animal models of wound healing (see, for example, chronic wound model: Hayward P G, Robson M C: Animal models of wound contraction. In Barbul A, et al: Clinical and Experimental Approaches to Dermal and Epidermal Repair: Normal and Chronic Wounds. John Wiley & Sons, New York, 1990; spinal cord injury model: Constantini, S., and Young, W. (1994), J. Neurosurg. 80, 97-111; Anderson, A. J., et al., Journal of Neurotrauma 21 (12), 1831-1846; brain injury model: Finnie, J., Aust Vet J. 2001 September;79(9): 628-33. Animal models of traumatic brain injury: a review). Smith (International Journal of Nanomedicine 2007:2(4), Perspectives on transdermal ultrasound mediated drug delivery) provides a comprehensive review of suitable ultrasound devices in Table 1, page 587-588.

ACCS is tested in various animal models of tissue regeneration (see, for example, tendon and ligament: Carpenter, J. E. and Hankenson, K. D., 2004, Biomaterials 25(9):1715-1722, Animal Models for Tissue Engineering Applications; cartilage: Chu C R, et al., Tissue Eng Part B Rev. 2010 February;16(1):105-15, Animal models for cartilage regeneration and repair; bone: Pearce, S. G., European Cells and Materials Vol. 14. Suppl. 1, 2007 (page 42), Animal Models for Bone Repair; Matos, M. A., et al., Journal of Orthopaedic Surgery and Research 2008, 3:4, Histomorphometric evaluation of bone healing in rabbit fibular osteotomy model without fixation; muscle: De Bari, C., et al., J Cell Biol. 2003 Mar. 17; 160(6): 909-918. Skeletal muscle repair by adult human mesenchymal stem cells from synovial membrane). Smith (International Journal of Nanomedicine 2007:2(4), Perspectives on transdermal ultrasound mediated drug delivery) provides a comprehensive review of suitable ultrasound devices in Table 1, page 587-588.

The present invention may be embodied in other specific forms without departing from the spirit or essential attributes thereof Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

What is claimed is:

1. A method of delivering Amnion-derived Multipotent Progenitor (AMP) cells into a body tissue comprising
a) mixing the AMP cells with an aqueous medium;
b) placing the aqueous medium/AMP cell mixture on the body tissue,
c) applying an ultrasound- delivering device to the body tissue; and
d) allowing the ultrasound-delivering device to emit ultrasound waves, wherein the ultrasound waves cause the AMP cells to be delivered into the body tissue.

2. A method of delivering Amnion-derived Cellular Cytokine Solution (ACCS) into a body tissue comprising
a) placing the ACCS on the body tissue,
b) applying an ultrasound- delivering device to the body tissue; and
c) allowing the ultrasound-delivering device to emit ultrasound waves, wherein the ultrasound waves cause the ACCS to be delivered into the body tissue.

3. The method of claim 1 or 2 wherein the body tissue is selected from the group consisting of epithelial tissue, connective tissue, muscle tissue, nervous tissue, and a combination thereof

4. The method of claim 3 wherein the epithelial tissue is on the external body surface or an internal body surface.

5. The method of claim 3 wherein the connective tissue is selected from the group consisting of areolar connective tissue, reticular connective tissue, adipose connective tissue, dense regular connective tissue, dense irregular connective tissue, elastic connective tissue, hyaline cartilage, elastic cartilage, fibrocartilage, compact bone, cancellous bone, and a combination thereof

6. The method of claim 3 wherein the muscle tissue is selected from the group consisting of skeletal muscle tissue, smooth muscle tissue, cardiac muscle tissue, and a combination thereof.

7. The method of claim 3 wherein the nervous tissue is selected from the group consisting of functional nervous tissue, supportive nervous tissue, and a combination thereof.

8. The method of claim 1 wherein the aqueous medium is selected from the group consisting of water, normal saline, Ringer's solution, cell culture medium, aqueous gels, salves and suspensions.

9. The method of claim 1 or 2 wherein the ultrasound-delivering device is a wand or a probe.

10. The method of claim 1 wherein the AMP cells are administered in combination with another agent and/or treatment modality.

11. The method of claim 2 wherein the ACCS is administered in combination with another agent and/or treatment modality.

12. The method of claim 1 wherein the AMP cells are treated such that they become genetically modified.

13. The method of claim 12 wherein the genetic modification is insertion of one or more genes into the cells.

14. The method of claim 13 wherein the insertion of one or more genes into the cells results in the formation of an induced pluripotent cell or an immortalized cell.
